(19) United States
(12) Patent Application Publication
(10) Pub. No.: US 2014/0025120 A1
(43) Pub. Date: Jan. 23, 2014

(54) MULTI-AXIAL BONE FASTENER AND SYSTEM

(71) Applicant: Robert Allan Farris, Cordova, TN (US)

(72) Inventor: Robert Allan Farris, Cordova, TN (US)

(73) Assignee: WARSAW ORTHOPEDIC, INC., Warsaw, IN (US)

(21) Appl. No.: 13/552,221

(22) Filed: Jul. 18, 2012

(51) Int. Cl. A61B 17/84; A61B 17/70

(52) U.S. Cl. 606/278; 606/300

(57) ABSTRACT

A bone fastener includes a receiver defining a longitudinal axis and an implant cavity. A base is connected to the receiver and includes a wall extending along the longitudinal axis and defining a lateral opening. The base further includes a bearing surface connected to the wall. The wall and the bearing surface define an interior cavity of the base, wherein the bearing surface has a first thickness adjacent the lateral opening and a second thickness adjacent a portion of the wall disposed opposite the lateral opening, the first thickness being greater than the second thickness. The bone fastener further includes a bone penetrating member having a proximal end and a distal end, the proximal end being disposable in the interior cavity of the base. Methods of use are disclosed.

### TECHNICAL FIELD

The present disclosure generally relates to medical devices, systems and methods for the treatment of musculoskeletal disorders, and more particularly to a spinal implant fixation system that employs a multi-axial bone fastener system to provide stabilization of vertebrae.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility. For example, after a disc collapse, severe pain and discomfort can occur due to the pressure exerted on nerves and the spinal column.

Non-surgical treatments, such as medication, rehabilitation and exercise can be effective, however, may fail to relieve the symptoms associated with these disorders. Surgical treatment of these spinal disorders include discectomy, laminectomy, fusion and implantable prosthetics. As part of these surgical treatments, spinal constructs such as vertebral rods are often used to provide stability to a treated region. During surgical treatment, one or more rods may be attached via fasteners to the exterior of two or more vertebral members. This disclosure describes an improvement over these prior art technologies.

### SUMMARY

Accordingly, a bone fastener is disclosed that includes a receiver defining a longitudinal axis and an implant cavity. A base is connected to the receiver and includes a wall extending along the longitudinal axis and defining a lateral opening. The base further includes a bearing surface connected to the wall. The wall and the bearing surface define an interior cavity of the base, wherein the bearing surface has a first thickness adjacent the lateral opening and a second thickness adjacent a portion of the wall disposed opposite the lateral opening, the first thickness being greater than the second thickness. The bone fastener further includes a bone penetrating member having a proximal end and a distal end, the proximal end being disposable in the interior cavity of the base.

In one embodiment, the bone fastener includes a bone penetrating member having a shank portion with a distal end, and a proximal end portion. A receiver includes a pair of upright spaced apart arms defining a lateral U-shaped channel, and a longitudinal axis. A base member is rotatably connected to the receiver. The base member has a wall extending along the longitudinal axis and defines an elongated lateral opening configured and dimensioned to receive the shank portion of the bone fastener. A bearing surface is connected to the wall. The wall and the bearing surface defines an interior cavity of the base in which the proximal end portion of the bone penetrating member is rotatably mounted. The bearing surface has a first thickness adjacent the lateral opening and a second thickness adjacent a portion of the wall disposed opposite the lateral opening. The first thickness being greater than the second thickness.

In one embodiment a vertebral rod fixation system is provided, which comprises at least one vertebral rod. A multi-axial bone fastener includes a distal shank portion and a proximal end portion. A receiver defines a longitudinal axis including a pair of upright spaced apart arms defining a lateral U-shaped channel which is configured for receiving the at least one vertebral rod. A base is rotatably connected to the receiver and has a circumferential wall extending along the longitudinal axis. An elongated lateral opening defined by the circumferential wall is configured to receive the shank portion. The base includes a circumferential fastener seat connected to the wall which is configured to support the head portion. The wall and the fastener seat define an interior cavity of the base. The fastener seat includes a reinforcement portion having a first thickness adjacent the lateral opening and connected to a second thickness adjacent a portion of the wall disposed opposite the lateral opening. The first thickness being greater than the second thickness such that a thickness of the reinforcement portion increases uniformly from the second thickness to the first thickness.

### BRIEF DESCRIPTION OF THE DRAWINGS

The present disclosure will become more readily apparent from the specific description accompanied by the following drawings, in which:

FIG. 1 is a side view, in part cross section, of one embodiment of the bone fastener of a system in accordance with the principles of the present disclosure;

FIG. 2 is a sectional side view of a base of the bone fastener shown in FIG. 1;

FIG. 3 is an elevational view of the system shown in FIG. 1 implanted with vertebrae; and

FIG. 4 is a sectional side view of the system and vertebrae shown in FIG. 3.

Like reference numerals indicate similar parts throughout the figures.

### DETAILED DESCRIPTION

The exemplary embodiments of bone fastener system and related methods of use disclosed are discussed in terms of medical devices for the treatment of musculoskeletal disorders and more particularly, in terms of a bone fastener that provides stabilization for treating a vertebral column.

In one embodiment, the bone fastener includes a gap in a screw seat such that the angle of rotation can be increased to allow bone penetrating member to rotate beyond the limit of the original seat opening, while maintaining and/or providing greater strength of the screw seat. In one embodiment, the bone fastener includes a counter sink that is machined off axis to the centerline resulting in additional material below a bearing surface for a screw head. It is envisioned that the material below the bearing surface has a minimum thickness at the furthest point from the gap and increases in thickness until the maximum thickness is achieved adjacent the gap. It is further envisioned that the additional material compensates for the lack of support in the gap. It is contemplated that the present bone fastener with increased angulation aids in difficult pathology and is easier to use due to the versatility in screw placement, which requires much less rod contouring for the surgeon.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other bone related applications, including those associated with diagnostics and therapeutics. It is contemplated that the disclosed bone fastener and system may be alternatively employed in a surgical treatment with a patient in a prone or supine position, and/or employ various surgical approaches to the spine, including anterior, posterior, posterior mid-line, medial, lateral, postero-lateral, and/or antero-lateral approaches, and in other body regions. The present disclosure may also be alternatively employed with procedures for treating the occipital, cervical, thoracic, lumbar, sacral, and pelvic regions of a spinal column. The bone fastener and system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “superior” and “inferior” are relative and used only in the context to the other, and are not necessarily “upper” and “lower”.

Further, as used in the specification and including the appended claims, “treating” or “treatment” of a disease or condition refers to performing a procedure that may include administering one or more drugs to a patient in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

The components of the bone fastener system can be fabricated from biologically acceptable materials suitable for medical applications, including metals, synthetic polymers, ceramics and bone material and/or their composites, depending on the particular application and/or preference of a medical practitioner. For example, the components of the bone fastener and system, individually or collectively, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyimide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of the bone fastener system may have material composites, including the above materials, to achieve various desired characteristics such as strength, rigidity, elasticity, compliance, biomechanical performance, durability and radiolucency or imaging preference. The components of the bone fastener system, individually or collectively, may also be fabricated from a heterogeneous material such as a combination of two or more of the above-described materials.

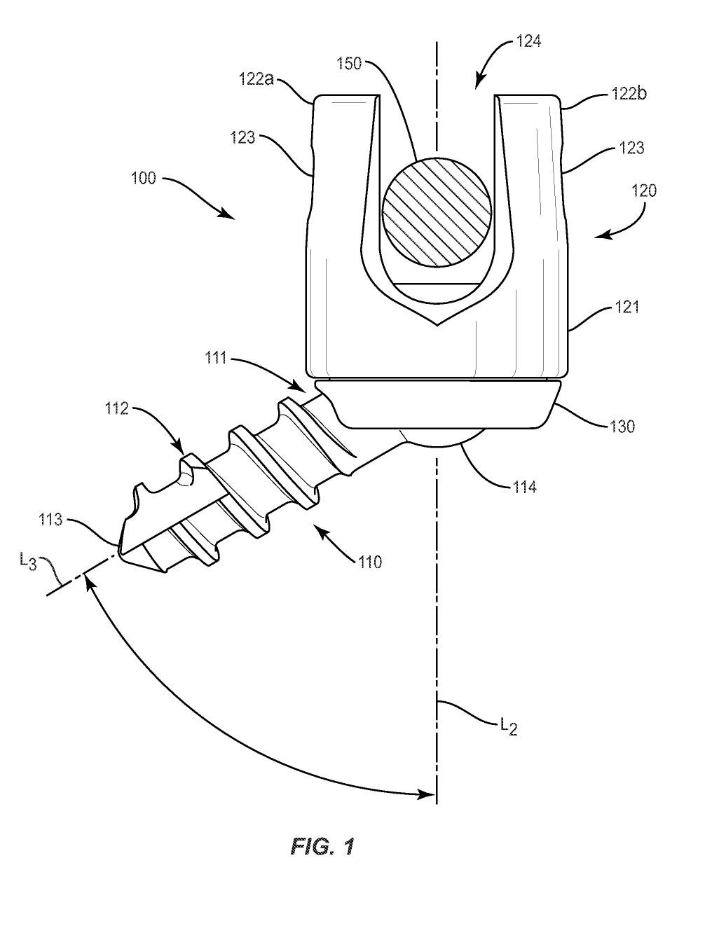

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications in the described devices, instruments, methods, and any further application of the principles of the disclosure as described herein are contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. The following discussion includes a description of a bone fastener system and related methods of employing the bone fastener and system in accordance with the principles of the present disclosure. Alternate embodiments are also disclosed. Reference will now be made in detail to the exemplary embodiments of the present disclosure, which are illustrated in the accompanying figures. Turning now to FIGS. 1-4, there is illustrated components of a bone fastener and system in accordance with the principles of the present disclosure.

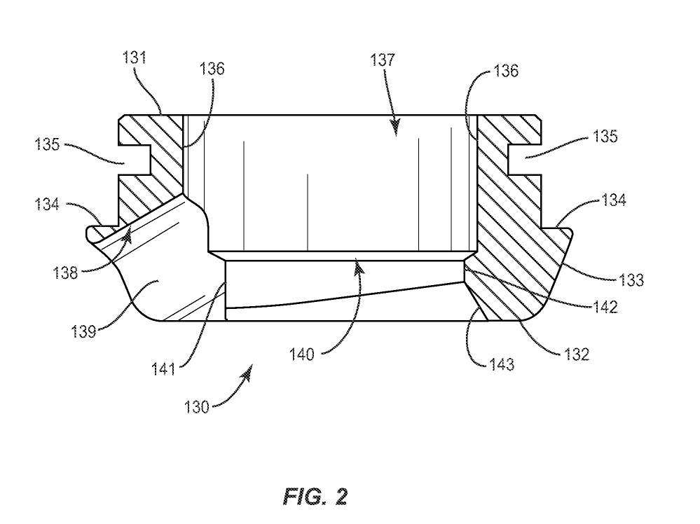

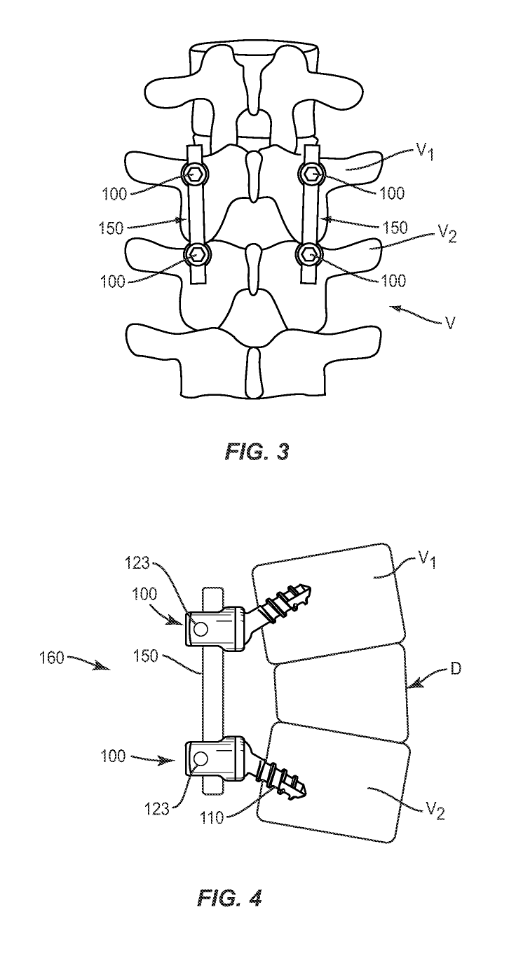

Bone fastener 100 is employed with a vertebral rod system, which is configured for attachment to bone, such as, for example, vertebrae V (as shown, for example, in FIGS. 3-4) during surgical treatment of a spinal disorder, examples of which are discussed herein. Referring to FIGS. 1 and 2, bone fastener 100 includes an elongated bone penetrating member 110 including a shank 111 having an outer surface 112 which is preferably threaded to a tip 113 to allow bone penetrating member 110 to function as a bone screw. Shank 111 has a cylindrical shaft configuration. Proximal end 114 includes a rounded head configured to fit within an interior cavity of a base, as discussed below, while allowing rotation and articulation of the bone penetrating member. A set screw (not shown) secures a rod 150 to the bone fastener 100. The bone fastener 100 may be part of a larger orthopedic system comprising a plurality of longitudinal members (e.g., rods, plates, etc.), a plurality of bone fasteners, and/or a plurality of connectors. In some embodiments, the bone fastener 100 is particularly suited for use in the spinal column. It will be understood that various types of fasteners or connectors (e.g. clamps) can be used in combination with the bone fastener 100 and rod 150.

Shank 111 defines a longitudinal axis L₁ and is configured for fixation with vertebrae V (FIGS. 3 and 4). It is contemplated that bone penetrating member 110 may include alternate bone fixation elements, such as, for example, a nail configuration, barbs, and/or expanding elements.

It is contemplated that bone penetrating member 110 can be variously dimensioned, for example, with regard to length, width, diameter and thickness. It is further contemplated that the respective cross-sectional geometry of member 110 may have various configurations, for example, round, oval, rectangular, irregular, consistent, variable, uniform and non-uniform. Member 110 may have a different cross-sectional area, geometry, material or material property such as strength, modulus or flexibility relative to shank 111.

Bone fastener 100 further includes a receiver 120 for a vertebral rod 150. More particularly, receiver 120 includes a body 121 defining a longitudinal axis L₂, and having upright arms 122a and 122b, which are spaced apart so as to define an implant cavity, or channel 124, to receive a vertebral rod 150 in a vertical orientation. It is understood that the channel 124 may be oriented at an angle to the longitudinal axis L₂. It is understood that the rod may have a number of desired lengths and diameters. In that regard, the width of the channel 124 in the current embodiment is substantially equal to the diameter of the rod member. In some embodiments, the width of the channel is slightly larger than the diameter of the rod, which allows easier insertion of the rod into the channel, allows for contouring of the rod, and also allows a variety of elongated member of differing sizes to be used with retainer 120. Generally, the rod 150 is positioned above the bottom portion of the channel 124 when in a locked position. However, in some embodiments the rod 150 may be seated within the bottom portion of the channel 124 when in a locked position. Thus, the bottom portion of the channel 124 may be shaped or otherwise include features to ensure secure placement of the elongated member.

The upright portions 122a and 122b of the receiver 120 include a recess or hole 123, which extends into or through the upright portions. The holes 123 are substantially aligned with one another and are substantially perpendicular to the channel 124. In some instances, the holes 123 are utilized for grasping by a surgical tool to facilitate positioning of the rod 150 into the bone fastener 100 within the patient. In an embodiment the upright portions 122a and 122b can have a tapered outer surface as they extend upwardly. This taper reduces the bulk and size of the receiver 120 allowing for easier handling. In that regard, a surgical instrument may engage the recess or holes 123 without substantially increasing the overall width needed to insert to the bone anchor assembly.

Bone fastener 100 further includes a base 130, which is rotatably mounted to the bottom of receiver 120. Base 130 includes a generally cylindrical disk shaped body having an upper end 131 and a lower end 132. Base 130 includes a circumferential exterior surface 133 at the lower end 132, a circumferential flange 134, which abuts a corresponding surface of the receiver 120, and a circumferential notch 135 adapted to engage a corresponding engagement member such as a ridge, snap ring, internal thread or other feature of the receiver 120 (not shown) to permit relative respective rotation without separation of the base 130 and the receiver 120. Base 130 includes an inner wall surface 136 defining an interior cavity 137 in which the rounded proximal head portion 114 of the bone penetrating member 110 is supported. The engagement between the proximal head 114 and the interior cavity 137 is such as to allow the bone penetrating member 110 to pivot such that an angle is formed between axis L₁ and axis L₂. Wall surface 138 of the base 130 defines an angular cutout 139 to increase the allowable angulation of the bone penetrating member 110 in relation to the receiver 120. Cutout 139 is configured and dimensioned so as to movably receive the shank 111 of the bone penetrating member, thereby allowing the bone penetrating member 110 to rotate beyond the limit of the original seat opening.

Base 130 also includes an interior circumferential bearing surface 140 circumferentially disposed about and configured to support the proximal head portion 114 of the bone penetrating member and provide a fastener seat. The bearing surface 140 is connected to the wall surface 136 to define an interior cavity of the base in which the proximal portion 114 of the bone penetrating member is disposed. The bearing surface includes a reinforcement portion having a first thickness 141 in the adjacent the lateral opening 139 and a second thickness 142 adjacent a portion of the wall 136 disposed opposite the lateral opening 139. First thickness 141 is greater than second thickness 142. In an embodiment the reinforcement portion thickness increases uniformly and/or gradually from the second thickness to the first thickness. Base 130 also possesses a circumferential beveled countersink surface 143 in the vicinity of lower end 132. In an embodiment the reinforcement portion can have a polished surface or a surface coated with a biologically inert material such as, e.g., fluorocarbon polymer to reduce friction.

As can be seen, the bone penetrating member 110 can be moved between a first position wherein the shank 111 is not engaged in lateral opening 139 and axis L₁ is aligned with axis L₂, and a second position wherein the shank 111 is maximally engaged in lateral opening 139 beyond the limit of the original seat opening.

The bone fastener 100 can be incorporated into a system 160 including at least one bone fastener 100, and vertebral rod 150 connected thereto. Typically, system 160 will include at least two bone fasteners 100 and a vertebral rod extending between and connected thereto.

In assembly, operation and use, the vertebral rod system 160 including bone fastener 100 is employed with a surgical procedure for treatment of a spinal disorder affecting a section of a spine of a patient, as discussed herein. The bone fastener 100 may also be employed with other surgical procedures. Bone fastener 100 is employed with a surgical procedure for treatment of a condition or injury of an affected section of the spine including vertebrae V, as shown in FIGS. 3-4. It is contemplated that the vertebral rod system 160 including bone fastener 100 is attached to vertebrae V for fusion and/or dynamic stabilization applications of the affected section of the spine to facilitate healing and therapeutic treatment.

In use, to treat the affected section of the spine, a medical practitioner obtains access to a surgical site including vertebrae V in any appropriate manner, such as through incision and retraction of tissues. It is envisioned that the vertebral rod system 160 including bone fastener 100 may be used in any existing surgical method or technique including open surgery, mini-open surgery, minimally invasive surgery and percutaneous surgical implantation, whereby vertebrae V is accessed through a micro-incision, or sleeve that provides a protected passageway to the area. Once access to the surgical site is obtained, the particular surgical procedure is performed for treating the spinal disorder. The vertebral rod system 160 including bone fastener 100 is then employed to augment the surgical treatment. The vertebral rod system 160 including bone fastener 100 can be delivered or implanted as a pre-assembled device or can be assembled in situ. The vertebral rod system may be completely or partially revised, removed or replaced, for example, replacing rod 150 and/or one or all of the components of bone fastener 100.

Vertebral rod 150 may be a rigid, rectilinear or optionally arcuate configuration. Alternatively, the rod 150 may be flexible, or a combination of flexible in parts and rigid in parts. A first bone fastener 100 is configured to attach an upper section of vertebral rod 150 to vertebra V₁. A second bone fastener 100 is configured to attach a lower section of vertebral rod 150 to vertebra V₂. Pilot holes are made in vertebrae V₁, V₂ for receiving first and second bone fasteners 100. Each bone penetrating member 110 of first and second bone fasteners 100 includes threaded bone engaging shank portion 112 that are inserted or otherwise connected to vertebrae V₁, V₂, according to the particular requirements of the surgical treatment. Each retainer 120 of first and second bone fasteners 100 includes channel 124 configured to receive and support rod 150, and a set screw, which is torqued into the receiver 120 to attach rod 150 in place with vertebrae V, as will be described. It is envisioned that vertebral rod 150 alternatively may have a semi-rigid or flexible configuration.

As shown in FIG. 3, the vertebral rod system includes two axially aligned and spaced rods 150, with end sections extending through channels 124 of receivers 120 of the bone fasteners 100. Set screws are torqued on the end portions of rods 150 to securely attach rods 150 with vertebrae V₁, V₂. An intervertebral disc D is disposed between vertebrae V₁ and V₂ (FIG. 4). Upon fixation of the vertebral rod system with vertebrae V, bone fasteners 100 are attached to vertebrae V for fusion and/or dynamic stabilization application of the affected section of the spine to facilitate healing and therapeutic treatment.

Bone fastener 100 may be employed as a bone screw, pedicle screw or multi-axial screw used in spinal surgery. It is contemplated that bone fastener 100 may be coated with an osteoconductive material such as hydroxyapatite and/or osteoinductive agent such as a bone morphogenic protein for enhanced bony fixation. Bone fastener 100 can be made of radiolucent materials such as polymers. Radiomarkers may be included for identification under x-ray, fluoroscopy, CT or other imaging techniques. Metallic or ceramic radiomarkers, such as tantalum beads, tantalum pins, titanium pins, titanium endcaps and platinum wires can be used, such as being disposed at the end portions of rod 150.

It is envisioned that the vertebral rod system described above including bone fastener 100 may be employed with a vertebral rod having an increased length providing the ability to extend over two or more intervertebral elements. It is contemplated that the configuration of the vertebral rod system may provide load sharing, dynamic and/or flexible stabilization over a plurality of intervertebral levels, including treated and untreated vertebral and intervertebral levels.

In one embodiment, the bone fastener includes an agent, which includes a bone growth promoting material, which may be disposed, packed or layered within, on or about the components and/or surfaces thereof. The bone growth promoting material, such as, for example, bone graft can be a particulate material, which may include an osteoconductive material such as hydroxyapatite and/or an osteoinductive agent such as a bone morphogenic protein (BMP) to enhance bony fixation of bone fastener 100 with the vertebrae V.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A bone fastener comprising:
a receiver defining a longitudinal axis and an implant cavity;
a base connected to the receiver and including a wall extending along the longitudinal axis and defining a lateral opening, the base further including a bearing surface connected to the wall, the wall and the bearing surface defining an interior cavity of the base, wherein the bearing surface has a first thickness adjacent the lateral opening and a second thickness adjacent a portion of the wall disposed opposite the lateral opening, the first thickness being greater than the second thickness; and
a bone penetrating member having a proximal end and a distal end, the proximal end being disposable in the interior cavity of the base.

2. The bone fastener of claim 1 wherein the base is rotatable relative to the receiver.

3. The bone fastener of claim 1 wherein the bearing surface is circumferentially disposed about the proximal end of the bone penetrating member.

4. The bone fastener of claim 1 wherein the lateral opening has an oblique orientation.

5. The bone fastener of claim 1 wherein the bearing surface has a countersink configuration.

6. The bone fastener of claim 1 wherein the first thickness is a maximum thickness of the bearing surface.

7. The bone fastener of claim 1 wherein the second thickness is a minimum thickness of the bearing surface.

8. The bone fastener of claim 1 wherein the bone penetrating member is movable within the lateral opening.

9. The bone fastener of claim 1 wherein the bearing surface includes a reinforced portion configured to support the proximal end of the bone penetrating member.

10. The bone fastener of claim 9 wherein the reinforced portion includes at least one of the first thickness and the second thickness.

11. The bone fastener of claim 1 wherein the bone penetrating member is disposed within the interior cavity in a configuration for rotation about the longitudinal axis and an axis transverse to the longitudinal axis.

12. The bone fastener of claim 1 wherein the distal end of the bone penetrating member is rotatable relative to the longitudinal axis of the receiver.

13. A multi-axial bone fastener comprising:
a bone penetrating member having a distal shank portion, and a proximal head portion;
a receiver defining a longitudinal axis and an implant cavity; and
a base rotatably connected to the receiver and having a wall extending along the longitudinal axis and defining an elongated lateral opening configured for disposal of the shank portion, the base including a bearing surface connected to the wall and being configured to support the head portion, the wall and the bearing surface defining an interior cavity of the base, wherein the bearing surface includes a reinforcement portion having a first thickness adjacent the lateral opening and a second thickness adjacent a portion of the wall disposed opposite the lateral opening, the first thickness being greater than the second thickness.

14. The bone fastener of claim 13 wherein the reinforcement portion gradually increases in thickness from the second thickness to the first thickness.

15. The bone fastener of claim 13 wherein the reinforcement portion thickness increases uniformly from the second thickness to the first thickness.

16. The bone fastener of claim 13 wherein the first thickness is circumferentially connected to the second thickness.

17. The bone fastener of claim 13 wherein the bearing surface has a countersink configuration.

18. The bone fastener of claim 13, wherein the first thickness is a maximum thickness of the bearing surface.

19. The bone fastener of claim 13 wherein the second thickness is a minimum thickness of the bearing surface.

20. A vertebral rod fixation system comprising:
at least one vertebral rod; and
a multi-axial bone fastener including a distal shank portion and a proximal end portion,
a receiver defining a longitudinal axis including a pair of upright spaced apart arms defining a lateral U-shaped channel configured for receiving the at least one vertebral rod and,
a base rotatably connected to the receiver having a circumferential wall extending along the longitudinal axis and defining an elongated lateral opening configured to receive the shank portion, the base including a circumferential fastener seat connected to the wall and being configured to support the head portion, the wall and the fastener seat defining an interior cavity of the base, wherein the fastener seat includes a reinforcement portion having a first thickness adjacent the lateral opening and connected to a second thickness adjacent a portion of the wall disposed opposite the lateral opening, the first thickness being greater than the second thickness such that a thickness of the reinforcement portion increases uniformly from the second thickness to the first thickness.
